(19) United States
(12) Patent Application Publication
(10) Pub. No.: US 2014/0073686 A1
(43) Pub. Date: Mar. 13, 2014

(54) COMPOSITIONS FOR MODULATING INVASION ABILITY OF A TUMOR AND METHODS THEREOF

(71) Applicants: Cheng-Chi CHANG, Taipei City (TW); Been-Ren LIN, Taipei City (TW); Yen-Ping KUO, Taipei City (TW)

(72) Inventors: Cheng-Chi CHANG, Taipei City (TW); Been-Ren LIN, Taipei City (TW); Yen-Ping KUO, Taipei City (TW)

(73) Assignee: NATIONAL TAIWAN UNIVERSITY, Taipei City (TW)

(21) Appl. No.: 13/613,733

(22) Filed: Sep. 13, 2012

(51) Int. Cl. A61K 31/7105; A61P 35/00; C40B 40/06; C12Q 1/68; G01N 33/574; C07H 21/02; C40B 30/04

(52) U.S. Cl. 514/44 R; 536/23.1; 506/9; 435/6.12; 435/7.9; 506/16

(57) ABSTRACT

The present invention provides a composition for modulating invasion ability of a tumor, comprising: an effective amount of an activator for a miRNA-mediated pathway or an effective amount of a modulating member in the miRNA-mediated pathway being a modulating member, and wherein the miRNA-mediated pathway is regulated by at least one miRNA selected from the group consisting of miR-346, miR-504 and miR-1179. The composition functions according to a novel model that an activator or a modulating member can regulate cellular invasion/migration of tumor via a miRNA-mediated pathway, and thereby can be a potential candidate of molecular drug to treat the tumor by modulating its invasion ability. A method for treating or preventing tumor invasion method is provided as well. Meanwhile, a method for detecting the invasive ability of a tumor in a subject and the kit thereof are also provided.

### FIELD OF THE INVENTION

The present invention relates to a composition for modulating invasion ability of a tumor according to a novel model that an activator or a modulating member can regulate cellular invasion/migration of tumor via a new discovered miRNA-mediated pathway. Also, the present invention relates to a treating or preventing method, a detecting method and a kit thereof based on above-mentioned model.

### BACKGROUND OF THE INVENTION

Oral squamous cell carcinoma (OSCC) is one of the 10 most frequent cancers worldwide with more than half a million patients being diagnosed (5% of all cancer) each year (Vokes et al., 1993; Haddad and Shin, 2008). In Taiwan, OSCC has been the sixth leading cause of death from cancer with nearly 5400 new cases and 2200 deaths per year, and the incidence of OSCC has increased sixfold in the past decade.

Despite evolution of management, the overall survival of patients has not improved significantly during the last 20 years, with 5-year survival rates between 45 and 50%. The major reason for poor prognosis is the propensity of OSCC to invade adjacent tissues. The rate of local recurrence at the primary site and regional recurrence at peripheral lymph node metastasis ranges from about 33-40% (Kademani, 2007; Wutzl et al., 2007; Jerjes et al., 2010).

However, the underlying molecular mechanisms in oral cancer are poorly understood, and thereby identifying genes and their pathways that are involved in the process of invasion and metastasis is a must for further understanding such a disease.

On the other side, CTGF (connective tissue growth factor; CCN2) is a 36-38 kDa secreted growth factor, which was initially discovered in 1991 as a secreted protein of human umbilical vascular endothelial cells (Soon et al., 2003). It is a multifunctional signaling modulator involved in various biologic or pathologic processes, such as angiogenesis, osteogenesis, renal disease, skin disorders and tumor development (Lau and Lam, 1999; Perbal, 2001; Yosimichi et al., 2001; Planque and Perbal, 2003). Recently, growing evidences have suggested that CTGF expression is highly associated with tumor progression, including breast cancer-induced bone metastasis (Kang et al., 2003), glioblastoma growth (Pan et al., 2002) and poor prognosis of esophageal cancer (Koliopanos et al., 2002).

Furthermore, CTGF also acts as a migratory inducer of breast cancer cells (Chen et al., 2007). In contrast, the inventor(s) of the present invention have previously demonstrated that CTGF inhibits the ability of colon cancer (Lin et al., 2005) and non-small cell lung cancer (Chang et al., 2004) cells to metastasize and invade the neighboring tissue.

The tumor-suppressive and metastasis-inhibitory effect of CTGF has also been demonstrated in OSCC: CTGF attenuates the growth of OSCC (Moritani et al., 2003), and a recent report showed that CTGF inhibits OSCC motility (Chuang et al., 2011). Therefore, these results suggest that the role of CTGF in different types of cancer may vary considerably, depending on the tissue involved. However, the impact of CTGF in regulating metastasis among different cancers and the underlying mechanisms are not fully elucidated.

Also, recent studies have revealed important roles of miRNAs and miRNA processing in tumorigenesis (Voorhoev et al., 2006; Kumar et al., 2007; Ma et al., 2007). Recent studies suggest the critical role of miRNAs in metastatic progression because one miRNA can regulate a set of functionally relevant genes simultaneously, which may reinforce the phenotypic change (Chan et al., 2005; Lim et al., 2005). But, how miRNA can regulate migration, invasion or metastasis in tumor cell and its clear pathway have not been found yet.

Accordingly, there is an urgent need to design a new therapy by modulating invasion ability of the tumor to treat or diagnose this major fatal cancer.

### SUMMARY OF THE INVENTION

The following only summarizes certain aspects of the invention and is not intended to be limiting in nature. These aspects and other aspects and embodiments are described more fully below. All references cited in this specification are hereby incorporated by reference in their entirety. In the event of a discrepancy between the express disclosure of this specification and the references incorporated by reference, the express disclosure of this specification shall control.

As embodied and broadly described herein, the compositions of the invention are used to treat diseases associated with abnormal and or unregulated invasion abilities. Disease states which can be treated by the methods and compositions provided herein include tumor. The invention is also directed to methods or kits of detecting these diseases by invasive ability thereof.

Accordingly, a first aspect of the invention is to provide a composition for modulating invasion ability of a tumor, comprising:

an effective amount of an activator for a miRNA-mediated pathway or an effective amount of a modulating member in the miRNA-mediated pathway, wherein the miRNA-mediated pathway is regulated by at least one miRNA selected from the group consisting of miR-346, miR-504 and miR-1179.

A second aspect of the invention provided herein is a method for treating or preventing tumor invasion in a subject in need thereof, the method comprising:

administering an effective amount of a composition according to the above-mentioned composition.

A third aspect of the invention provided herein is a method for detecting the invasive ability of a tumor in a subject, comprising the steps of:

determining an expression level of an activator for a miRNA-mediated pathway or a modulating member in the miRNA-mediated pathway in a sample obtained from the subject, wherein the miRNA-mediated pathway is regulated by at least one miRNA selected from the group consisting of miR-346, miR-504 and miR-1179;

comparing the expression level of the activator or the modulating member in the sample with a standard level of the activator or the modulating member in the sample; and

whereby a difference between the expression level and the standard level indicates the invasive ability of the tumor.

A fourth aspect of the invention provided herein is a kit for detecting the invasive ability of a tumor in a subject, the kit comprising:

an activator for a miRNA-mediated pathway or a modulating member in the miRNA-mediated pathway being a modulator in a sample obtained from the subject, wherein the miRNA-mediated pathway is regulated by at least one miRNA selected from the group consisting of miR-346, miR-504 and miR-1179; and

an instruction for detecting the invasive ability of a tumor in a subject according to above-mentioned method.

In some embodiments, the activator may be a CTGF gene product. In some specific embodiments, the CTGF gene product may be a transcript, a polypeptide, or a protein.

In some embodiments, the modulating member of miRNA-mediated pathway may further comprise one or more upstream or downstream target gene product(s) involved in, which may be the modulator. In another embodiment, the modulating member may be a miRNA downstream target gene product such as FOXP1, PITPNA, CEP170 or the combination thereof.

In other embodiments, the modulating member may be a miRNA which can mediate or regulate above-mentioned pathway. In another embodiment, the modulating member may be miR-346, miR-504, miR-1179 or the combination thereof.

The details of one or more embodiments of the invention are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 illustrated the result of Expression of CTGF-inhibited invasion and migration abilities in human oral cancer cells according to one embodiment of the present invention, wherein

(a) Left: the invasion activity of human oral cancer cell lines were measured in vitro with Boyden chamber after 48 h. Right: endogenous CTGF expression was analysis by reverse transcription—PCR and western blot.

(b) Upper: the invasion ability of SAS cells treated with different doses of rCTGF (*P<0.05, **P<0.01). Lower: the migration ability of SAS cells treated with different doses of rCTGF. *P<0.05; **P<0.01.

(c) Upper: western blot analysis of CTGF protein expression in SAS cells after transiently transfected with different doses of CTGF expression plasmid for 48 h. Second: transfected cells were subcultured into Transwell for 48 h, and invaded cells were stained and counted (*P<0.05). Lower: transfected cells were subcultured into each wound-produced culture insert, and migrated cells toward the wounded area was observed and photographed every six hours (*P<0.05, **P<0.01).

(d) Upper: western blot analysis of CTGF expression in mock-transfected SAS (SAS/NEO) cells, CTGF-overexpressed mixture clone SAS/CTGF-M3, and single clone SAS/CTGF-C2, SAS/CTGF-C7, and SAS/CTGF-C28 cells (upper panel). Second: the invasion ability of each clone was measured in vitro with Boyden chamber after 48 h (*P<0.05, **P<0.01, ***P<0.005). Lower: transfectant cell migration toward the wounded area was observed and photographed every six hours (*P<0.05, **P<0.01).

(e) Western blot analysis of CTGF expression in TW2.6 cells transfected with various dosage of short-interference RNA-mediated knockdown of CTGF (siCTGF) nucleotide (upper panel). Invasion activity of CTGF-knockdown cells was measured after 48 h (lower panel, *P<0.05).

FIG. 2 illustrated miRNA expression results regulated by CTGF in human oral cancer cells, wherein

(a) Quantitative RT-PCR analysis of miR-346, miR-504 and miR-1179 expression in CTGF-overexpressed M3 clone and mock-transfected (SAS/Neo) cells (upper panel) and rCTGF treatment at indicated time in SAS cells (*P<0.05, lower panel).

(b) Quantitative RT-PCR analysis of transient-transfected miR-346 expression plasmids in SAS/CTGF-M3 stable clones after 48 h (upper panel). The invasion ability of transient-transfected miR-346 expression plasmids in SAS/CTGF-M3 stable clones (lower panel).

(c) Migration ability of transient-transfected miR-346 expression plasmids in SAS/CTGF-M3 stable clones (upper panel). The migrated cells toward the wounded area were counted at indicated time (lower panel).

FIG. 3 illustrated the overexpression results of miR-504 enhances invasion and migration abilities in CTGF transfectants, wherein

(a) Quantitative RT-PCR analysis of stably transfected miR-504 expression plasmids in CTGF-overexpressed mixture clone (SAS/M3-miR-504-1, -2 and -3) compared with vector control (SAS/CTGF-M3/Neo) (**P<0.01, ***P<0.005, left panel). Invasion activity of miR-504 stable transfectant in CTGF-stable clone versus SAS/M3-Neo (control) clones after 48 h (**P<0.01, ***P<0.005, right panel).

(b) Migration abilities of SAS/M3-miR-504 stable clones (SAS/M3-miR-504-1, -2 and -3) and vector control cells (**P<0.01).

FIG. 4 illustrated the determined results that FOXP1 is the downstream target gene of the CTGF-regulated miR-504 signalling pathway in human oral cancer cells, wherein

(a) Upper: RT-PCR analysis of PITPNA, FOXP1, CEP170 and CTGF in CTGF transfectants (SAS/CTGF-M3) versus control (SAS/Neo), and CTGF-knockdowned cells (TW2.6-shCTGF-M1) versus control (TW2.6-Neo). Lower: quantitative RT-PCR analysis of PITPNA, FOXP1 and CEP170 mRNA expression in SAS/CTGF-M3 versus SAS/Neo, and TW2.6-shCTGF-M1 versus TW2.6-Neo. *P<0.05.

(b) RT-PCR (upper) and western blot (second) of FOXP1 in SAS/CTGF-M3 transiently transfected with the indicated dosage of miR-504. Lower: quantitative analysis of FOXP1 mRNA expression in SAS/CTGF-M3 transfected with different doses of miR-504.

(c) Upper: diagram depicting the 30UTR reporter assay. Lower: the luciferase activities of these reporters were examined with control and miR-504 for FOXP1, PITPNA and CEP170. *P<0.05.

(d) Upper: western blot of FOXP1 and quantitative RT-PCR analysis of FOXP1 mRNA expression in SAS/CTGF-M3 and control clones transiently transfected with a short-hairpin FOXP1 plasmid or a control vector. Lower: the invasion abilities of these clones measured by the modified Boyden chamber. *P<0.05, **P<0.01.

(e) The migration ability of SAS clones measured by a wound-produced culture insert. **P<0.01.

FIG. 5 illustrated the result that CTGF-miR-504-FOXP1 axis suppresses metastasis of orthotopically implanted tumor, wherein

(a) Kaplan-Meier plots of overall survival in experiment SCID mice orthotopically injected (buccal mucosa) with SAS/Neo, SAS/CTGF-M3, C7, and C28 clones. n=5 per group.

(b) Incidence of cervical lymph nodes of mice injected with SAS/Neo, SAS/CTGF-M3, C7 and C28. *P<0.05.

(c) Kaplan-Meier plots of overall survival in experiment SCID mice orthotopically injected (buccal mucosa) with SAS/Neo, SAS/CTGF-M3, SAS/CTGF-M3 reconstituted with miR-504 (SAS/CTGF-M3/miR-504), and co-transfection of miR-504 and FOXP1 in SAS/CTGF-M3 cells (SAS/CTGF-M3/miR-504/FOXP1). n=5 per group.

(d) Incidence of cervical lymph nodes of mice injected with SAS/Neo, SAS/CTGF-M3, SAS/CTGF-M3/miR-504, and SAS/CTGF-M3/miR-504/FOXP1. *P<0.05.

FIG. 6 illustrated the result of clinical importance of CTGF-miR-504-FOXP1 axis in OSCC patients, wherein

(a) Correlation between CTGF mRNA expression and tumor stage (I, II versus III, IV) in OSCC patients.

(b) Kaplan-Meier survival curves of OSCC cases divided by CTGF mRNA expression.

(c) Correlation between miR-504 expression and tumor stage (I, II versus III, IV) in OSCC patients.

(d-f) Correlation between CTGF/miR-504 (d), miR-504/FOXP1 (e) and CTGF/FOXP1 (f) in OSCC patients. The P-values were shown in each panel, and the correlation coefficients γ were shown in panels d-f.

FIG. 7 illustrated a proposed model CTGF-mediated inhibition of migration and invasion in human oral cancer cells through miR-504/FOXP1 signal pathway.

### DETAILED DESCRIPTION

### I. Embodiments

In describing and claiming the invention, the following terminology will be also used in accordance with the definitions set forth below.

In the first aspect of the invention, a composition for modulating invasion ability of a tumor is provided. The composition comprises an effective amount of an activator for a miRNA-mediated pathway or a modulating member in the miRNA-mediated pathway, wherein the miRNA-mediated pathway is regulated by at least one miRNA selected from the group consisting of miR-346, miR-504 and miR-1179. The composition provided herein is a new composition for treating a tumor by modulating its invasion ability via a new discovered miRNA-mediated pathway.

As described herein, the term “MicroRNA (miRNA)” refers to which is single-stranded RNA molecules that regulate gene expression. miRNAs are small noncoding regulatory RNAs ranging in size from 17 to 25 nucleotides. miRNAs are processed from primary transcripts known as pri-miRNA, to short stem-loop structures called pre-miRNA, and finally to functional miRNA. Mature miRNA molecules are partially complementary to one or more messenger RNA molecules, and their primary function is to down-regulate gene expression.

Thus, the term “miRNA-mediated pathway” refers to a signaling pathway regulated by miRNA and involved in a number of cellular processes, such as cell growth, proliferation, differentiation, migration, survival, intracellular trafficking, metabolism, invasion, and angiogenesis. In some embodiments, members of the miRNA-mediated pathway include, but are not limited to, miRNA(s) (e.g., miR-346, miR-504 and miR-1179) and miRNA downstream target gene(s) (e.g., FOXP1). In a preferred embodiment, the miRNA-mediated pathway may be a miR-504/FOXP1 pathway.

The terms “regulate”, “regulation” or variants thereof refer to influencing the specific level, the reacted rate or the activity of a molecule. In some embodiments of the present invention, the miRNA may regulate the miRNA-mediated pathway by initiate, block, or accelerate a reaction of a compound related to the miRNA-mediated pathway. Alternatively, the miRNA may regulate the miRNA-mediated pathway by either inhibiting (e.g., decreases or downregulates) or activating (e.g., increases or upregulates) expression or activity of a compound involved in the miRNA-mediated pathway.

The terms “modulate” and variants thereof (e.g., “modulating”), as described herein, are meant to either inhibit (e.g., decreases or downregulates) or activate (e.g., increases or upregulates) expression or activity of a molecule. For example, the molecule may be related to the miRNA-mediated pathway, or be involved in the miRNA-mediated pathway (such as miRNA or a miRNA regulated target).

As described herein, the term “a modulating member” or “modulator” include any type of molecule that may either inhibit or activate expression or activity of a molecule in a signal pathway (e.g., a miRNA-mediated pathway as described herein). In some embodiments, the modulating member may be selected from the group consisting of miR-346, miR-504 and miR-1179. Preferably, in certain embodiments, the modulating member may be miR-504. In other embodiments, the modulating member may be a miRNA downstream target gene product (e.g., a transcript or a protein). In some embodiments, the modulating member may be a gene product selected from the group consisting of FOXP1, PITPNA and CEP170. In a preferred embodiment, the modulating member may be a gene product of FOXP1.

In a specific embodiment, when the miRNA-mediated pathway is a miR-504/FOXP1 pathway, the modulating member may be miR-504 or a gene product of FOXP1. As described herein, FOXP1 is a member of ‘forkhead’ (Fox) transcription factors, which have critical roles in immune responses, organ development and cancer pathogenesis (Carlsson and Mahlapuu, 2002; Katoh, 2004). However, the role of FOXP1 in cancer migration has never been addressed before the present invention.

As described herein, the term “activator” includes any type of molecule that stimulates, increases, opens, activates, facilitates, enhances or up-regulates the expression or activity of a target gene or protein. An activator can be any type of compound, such as a small molecule, antibody or antisense compound. In some embodiments, the target gene or protein is a modulating member of the miRNA-mediated pathway, preferably, a miR-504-mediated pathway. In some embodiments, the activator may be a CTGF gene product such as a transcript, a polypeptide, or a protein. In a specific embodiment, the CTGF gene product may be a recombinant CTGF protein (rCTGF).

The tumor described herein may be any neoplastic cell growth and proliferation, whether malignant or benign, and any pre-cancerous and cancerous cells and tissues that may be regulated by the miRNA-mediated pathway. In some embodiments, the tumor may be squamous cell carcinoma (SCC). In other embodiments, the tumor may be oral cancer. In some specific embodiment, the tumor may be OSCC. If the tumor is OSCC, in a preferred embodiment, the miRNA-mediated pathway is a miR-504/FOXP1 pathway, while the modulating member may be miR-504, a gene product of FOXP1 or both.

As used herein an effective amount” or a “therapeutically effective amount” refers to a nontoxic but sufficient amount of a specific substance to provide the desired effect. For example, one desired effect of the activator would be the activation of the miRNA-mediated pathway. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The second aspect of the invention provides a method for treating or preventing tumor invasion in a subject in need thereof. The method comprising: administering an effective amount of a composition according to the above-mentioned composition.

“Administration” and variants thereof (e.g., “administering” a compound) in reference to the composition of the invention means introducing the composition into the system of the animal in need of treatment. When the composition is provided in combination with one or more other active agents (e.g., surgery, radiation, chemotherapy, etc.), “administration” and its variants are each understood to include concurrent and sequential introduction of the composition thereof and other agents.

The introduction of a composition into a subject is by a chosen route. For example, if the chosen route is intravenous, the composition is administered by introducing the composition into a vein of the subject. In some embodiments, the route of administration of a pharmaceutical composition is oral, topical or systemic.

As used herein, the term “treating” includes prophylaxis of the specific disorder or condition, or alleviation of the symptoms associated with a specific disorder or condition and/or preventing or eliminating said symptoms.

In some embodiments, the composition may further comprise a pharmaceutically acceptable carrier. In other embodiments, the composition may further comprise a pharmaceutically acceptable salt.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the US Federal government or listed in the US Pharmacopeia for use in animals, including humans.

As used herein, the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

Any ordinary skilled person in this art may know how to select a proper pharmaceutically acceptable carrier, a pharmaceutically acceptable salt thereof for implementing this invention without undue experimentation.

As used herein, the term “subject” or “patient” for the purposes of the present invention includes humans and other animals, particularly mammals, and other organisms. Thus the methods are applicable to both human therapy and veterinary applications. In another embodiment the patient is a mammal, and in another embodiment the patient is human.

In the third aspect of the invention, a method for detecting the invasive ability of a tumor in a subject is provided herein. The method comprises the steps of: determining an expression level of an activator for a miRNA-mediated pathway or a modulating member in the miRNA-mediated pathway in a sample obtained from the subject, wherein the miRNA-mediated pathway is regulated by at least one miRNA selected from the group consisting of miR-346, miR-504 and miR-1179; comparing the expression level of the activator or the modulating member in the sample with a standard level of the activator or the modulating member in the sample; and whereby a difference between the expression level and the standard level indicates the invasive ability of the tumor.

In some specific embodiments, the miRNA-mediated pathway may be a miR-504-mediated pathway.

In some embodiments, the activator may be a CTGF gene product. In a specific embodiment, the CTGF gene product may be a recombinant CTGF protein (rCTGF).

In some embodiments, the modulating member is a miRNA which mediates miRNA-mediated pathway and is selected from the group consisting of miR-346, miR-504 and miR-1179. In some specific embodiments, the modulating member is miR-504. In other embodiments, the modulating member a miRNA downstream target gene product involved in the miRNA-mediated pathway selected from the group consisting of FOXP1, PITPNA and CEP170. In some specific embodiments, the modulating member may be FOXP1. In a preferred embodiment, the modulating members are both miR-504 and FOXP1.

In some embodiments, the tumor may be oral cancer. In some specific embodiment, the tumor may be OSCC. If the tumor is OSCC, in a preferred embodiment, the miRNA-mediated pathway is a miR-504/FOXP1 pathway, while the modulating member may be miR-504, a gene product of FOXP1 or both.

The term “sample” is used herein in its broadest sense. Samples may be derived from any source, for example, from bodily fluids, secretions, or tissues including, but not limited to, saliva, blood, urine, and organ tissue (e.g., biopsied tissue); from chromosomes, organelles, or other membranes isolated from a cell; from genomic DNA, cDNA, RNA, mRNA, etc.; and from cleared cells or tissues, or blots or imprints from such cells or tissues. A sample can be in solution or can be, for example, fixed or bound to a substrate. A sample can refer to any material suitable for testing for the presence of CTGF or suitable for screening for molecules that bind to CTGF or fragments thereof. Methods for obtaining such samples are within the level of skill in the art.

The term “determining an expression level” refers to detecting the level of mRNA or protein expression which means to quantify the amount of a particular mRNA or protein (such as FOXP1 or CTGF protein or miR-504) present in a sample. Detecting expression of mRNA or protein can be achieved using any method known in the art or described herein, such as by RT-PCR (for mRNA) or Western blot (for protein).

In the fourth aspect of the invention, a kit is provided for detecting the invasive ability of a tumor in a subject. The kit comprises an activator for a miRNA-mediated pathway or a modulating member in the miRNA-mediated pathway being a modulating member in a sample obtained from the subject; and an instruction for detecting the invasive ability of a tumor in a subject according to above-mentioned method, wherein the miRNA-mediated pathway is regulated by at least one miRNA selected from the group consisting of miR-346, miR-504 and miR-1179.

The kit may further include a variety of containers, e.g., vials, tubes, bottles, and the like. Preferably, the kits will also include instructions for use. In some embodiments, the kit may further comprise instructions for detecting the invasive ability of a tumor in a subject according to above-mentioned method.

In some specific embodiments, the miRNA-mediated pathway may be a miR-504-mediated pathway.

In some embodiments, the activator may be a CTGF gene product. In a specific embodiment, the CTGF gene product may be a recombinant CTGF protein (rCTGF).

In some embodiments, the modulating member is a miRNA which mediates miRNA-mediated pathway and is selected from the group consisting of miR-346, miR-504 and miR-1179. In some specific embodiments, the modulating member is miR-504. In other embodiments, the modulating member is a miRNA downstream target gene product involved in the miRNA-mediated pathway selected from the group consisting of FOXP1, PITPNA and CEP170. In some specific embodiments, the modulating member may be FOXP1. In a preferred embodiment, the modulating members are both miR-504 and FOXP1.

In some embodiments, the tumor may be oral cancer. In some specific embodiment, the tumor may be OSCC. If the tumor is OSCC, in a preferred embodiment, the miRNA-mediated pathway is a miR-504/FOXP1 pathway, while the modulating member may be miR-504, a gene product of FOXP1 or both.

### II Examples

Materials and Methods

Cell Lines, Reagent and Culture

Five human OSCC cell lines were used, including CA9-22, CAL-27, HSC-3, SAS and TW2.6. Recombinant CTGF is purchased from BioVender (Heidelberg, Germany).

miRNA Microarray Analysis

Total RNA was isolated from SAS/CTGF-M3 cells and vector control cells with Trizol (Invitrogen Corporation, Carlsbad, Calif., USA). Amplification and hybridization were performed according to the manufacturer's protocol (Illumina, Inc., San Diego, Calif., USA). Illumina human V6 array was used for gene expression analysis. The raw data of the spot density was extracted from Illumina BeadStudio software and deposited into the Gene Expression Omnibus database (accession number GSE9742). Sample clustering analysis and raw data filtering (P<0.05) were performed. Quantile normalization was performed on the filtering data, followed by one-way analysis of variance to identify significant genes.

Reverse Transcription and Taqman-Based Quantitative Reverse Transcription—PCR Assays of miRNA Expression

Expressions of mature miRNAs were analyzed by TaqMan miR Assay (Applied Biosystems, Foster City, Calif., USA). Briefly, complementary DNA was synthesized from total RNA (100 ng) using the TaqMan MicroRNA Reverse Transcription Kit (Applied Biosystems). The reactions were incubated first at 16° C. for 30 min and then at 42° C. for 30 min followed by inactivation at 85° C. for 5 min. The reactions were then incubated in a 96-well plate at 50° C. for 2 min, 95° C. for 10 min, followed by 40 cycles of 95° C. for 15 s and 60° C. for 1 min using the ABI Prism 7000 Sequence Detection System (Applied Biosystems). Relative quantification of gene expression was performed using the endogenous control gene (RNU-6B). The threshold cycle (CT) was defined as the fractional cycle number at which the fluorescence passed the fixed threshold. Relative expression was calculated using the comparative CT method.

Western Blot Analysis

Proteins in the total cell lysate (55 μg of protein) were separated on a 10% sodium dodecyl sulfate-polyacrylamide gel electrophoresis gel and electrotransferred onto a polyvinylidene difluoride membrane (Immobilon-P membrane; Millipore, Bedford, Mass., USA). After the blot was blocked with a solution of 5% skim milk, 0.1% Tween 20 and Tris buffer saline Tween20 (TBST), membrane-bound proteins were probed with primary antibodies against CTGF and α-tubulin (Santa Cruz Biotechnology, Santa Cruz, Calif., USA). The membrane was washed and then incubated with horseradish peroxidase-conjugated secondary antibodies for 60 min. Antibody-bound protein bands were detected with enhanced chemiluminescence reagents (Amersham Pharmacia Biotech, Piscataway, N.J., USA) and exposed to Kodak X-Omat Blue autoradiography film (Perkin Elmer Life Sciences, Boston, Mass., USA).

Plasmid Construction

Single strands of miRNA were annealed to form double-strand miRNA DNA and inserted into the BLOCK-iT Pol II miR RNAi expression vector, (pcDNA6.2-GW/EmGFP-miR; Invitrogen Corporation). SAS/CTGF-M3 cells were grown overnight and transfected with plasmid pcDNA6.2-GW/EmGFP-miR-504 to express miR-504 or empty plasmid (negative control). Blasticidin (10 μg/ml) was used to select for stable clones.

Plasmids and Transient Transfection

For plasmid transfection, cells were transfected with 1-3 μg plasmids using Lipofectamine 2000 reagent (Invitrogen Corporation) in Opti-MEM medium (Invitrogen Corporation) for 4-18 h, after which the medium was replaced with fresh complete medium. After 24-48 h, the transfected cells were harvested and subjected to invasion assay, wound-healing migration assay and western blot analysis.

Selection of Stably Transfected Clones

Purified plasmid DNA (3 μg) was transfected into SAS cells with Lipofectamine 2000 reagent (Invitrogen Corporation). At 24 h after transfection, stable transfectants were selected with 800-1200 μg/ml Neomycin (G418; Life Technologies Corporation, Calsbad, Calif., USA). Thereafter, the selection medium was replaced every 2 days. After 2 weeks of selection in G418, resistant clones were isolated and allowed to proliferate in medium containing G418 100 μg/ml. Integration of transfected plasmid DNA was confirmed by reverse transcription—PCR and western blot analysis.

In Vitro Cell Growth Assay

In all, 2×10⁴ cells were seeded in a 24-well plate, and further cultured for 5 days. Cell number was determined at regular intervals using 3-(4,5-dimethylthiazol-2-yl)-2,5-diphenyltetrazolium bromide (MTT) assay.

Boyden Chamber Assays

For invasion assays, modified Boyden chambers with filter inserts (pore size, 8 μm) coated with Matrigel (20 μg; Collaborative Biomedical, Becton Dickinson Labware, Benford, Mass., USA) in 24-well dishes was used. Approximately, 2×10⁴ cells in 100 μl of 2% fetal bovine serum Dulbecco's modified Eagle's medium were placed in the upper chamber, and 900 μl of the same medium was placed in the lower chamber. After 48 h in culture, cells were fixed in methanol for 15 min and then stained with 0.05% crystal violet in phosphate-buffered saline for 15 min. Cells on the upper side of the filter were removed with cotton swabs, and the filters were washed with phosphate-buffered saline. Cells on the underside of the filters were viewed and counted under a microscope.

Wound-Healing Migration Assay

In all, 75 μl of 5×10⁵ cells per 1 ml was applied into each wound-produced culture insert (400±50 μm; ibidi Gmbh, Germany) and incubated overnight. Culture inserts were removed after appropriate cell attachment. Cells were washed twice with phosphate-buffered saline and serum-free medium was added. Cell migration toward the wounded area was observed and photographed.

Real-Time Quantitative RT-PCR

Complementary DNA was generated using the Taqman reverse transcription kit and poly dT primer according to the manufacturer's instructions. The complementary DNA was used as template in real-time quantitative PCR reactions with CTGF and β-actin-specific primers using the ABI Prism 7000 Sequence Detection System (Applied Biosystems) at 95° C. for 10 min, followed by 40 cycles of 95° C. 15 s and 60° C. for 1 min. Target gene expression was normalized between different samples based on the values of β-actin RNA expression. The primers used in quantitative reverse transcription-PCR were listed in Table 1.

TABLE 1
RT-PCR primer sequence

| Gene | | Annealing | | |
|---|---|---|---|---|
| Names | Cycle | Tm (° C.) | strand | Sequence |
| CTGF | 28 | 55 | F(SEQ ID | GCTTACCGACTGGAAGACACGTT |
| | | | NO.: 1) | |
| | | | R(SEQ ID | TCATGCCATGTCTCCGTACATC |
| | | | NO.: 2) | |
| PITPNA | 31 | 59 | F(SEQ ID | CCTGTGGGGAGCGGGGATGA |
| | | | NO.: 3) | |
| | | | R(SEQ ID | CTGAGTGGCAGCACCAGCCC |
| | | | NO.: 4) | |
| CEP170 | 31 | 57 | F(SEQ ID | ACAGGTGCAGGGCATGCTTCA |
| | | | NO.: 5) | |
| | | | R(SEQ ID | TCTACTCCAAACACAACAGCTTGGT |
| | | | NO.: 6) | C |
| FOXP1 | 34 | 55 | F(SEQ ID | GCCGATTCATTCCACGCAGCAGTA |
| | | | NO.: 7) | |
| | | | R(SEQ ID | CCACACCCGTTATCGCAGAGCAC |
| | | | NO.: 8) | |
| GAPDH | 30 | 55 | F(SEQ ID | GAAGGTGAAGGTCGGAGTC |
| | | | NO.: 9) | |
| | | | R(SEQ ID | CAGGAGGCATTGCTGATGA |
| | | | NO.: 10) | |

Animal Metastasis Experiment

In all, 6-8 weeks old female C.B.17-SCID mice (Experimental Animal Center in Medical College of National Taiwan University, Taiwan) were caged in groups. Mice were randomized to different groups receiving orthotopic injection of tumor cells into buccal mucosa (density of 10⁵ cells in 20 μl phosphate-buffered saline; n=5 for each group). For lymph node metastatic models, cervical lymph nodes were excited, counted, and examined with hematoxylin and eosin pathological examination.

Luciferase Reporter Assay

The 3′ UTR of human PITPNA, FOXP1 and CEP170 were amplified using PCR and cloned into a pMIR-Report vector. These constructs (1 ng) were independently co-transfected with 3 μg of control plasmid or plasmids expressing miR-504 and β-gal plasmid (0.2 ng) into 293T cells. Luciferase activity was measured 48 h after transfection using the dual-luciferase reporter assay system (Promega Corporation, Madison, Wis., USA).

OSCC Tumor Samples and Clinical Data Collection

OSCC specimens were collected at the time of surgery from previously untreated patients who underwent surgical resection at the National Taiwan University Hospital. Organization samples were snap-frozen immediately and stored at −80° C. The histologic identification of oral cancer was determined as recommended by the World Health Organization. Tumor size, local invasion and lymph node metastasis were determined at pathologic examination. The final disease stage was determined by a combination of surgical and pathologic findings, according to the current tumor-node-metastasis staging system for oral cancer. Follow-up data were obtained from the patients' medical charts and from our tumor registry service. The survival time of patients was calculated from the date of surgery to the date of death. The relapse time was calculated from the date of surgery to the date of local recurrence or distant metastasis.

Statistical Analysis

The background data of the patients with OSCC were compared using the Mann-Whitney test for scale variables (expressed as mean s.d.) and Fisher's exact test for nominal variables. Survival data were analyzed using the Kaplan-Meier method. Kaplan-Meier curves were compared by a log-rank test. P-values were two-sided and the significance level was 0.05.

### Example 1

### The Relationship Between CTGF Expression with Invasion and Migration Potential of Human Oral Cancer Cells

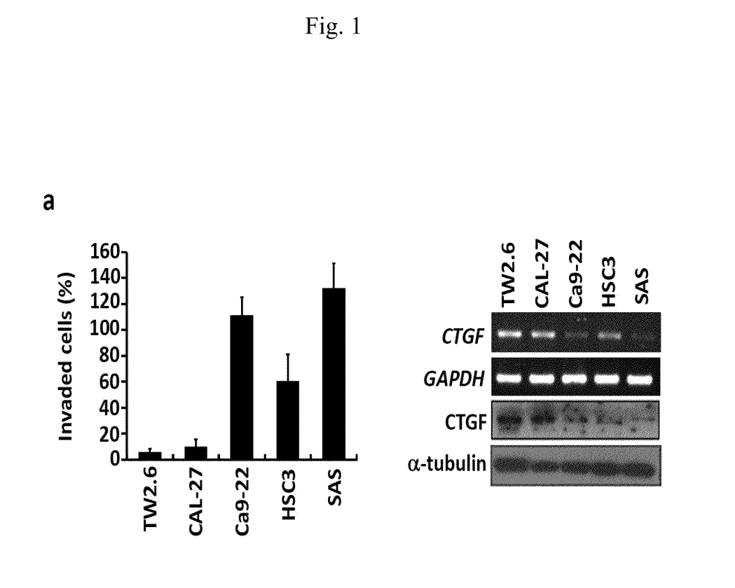

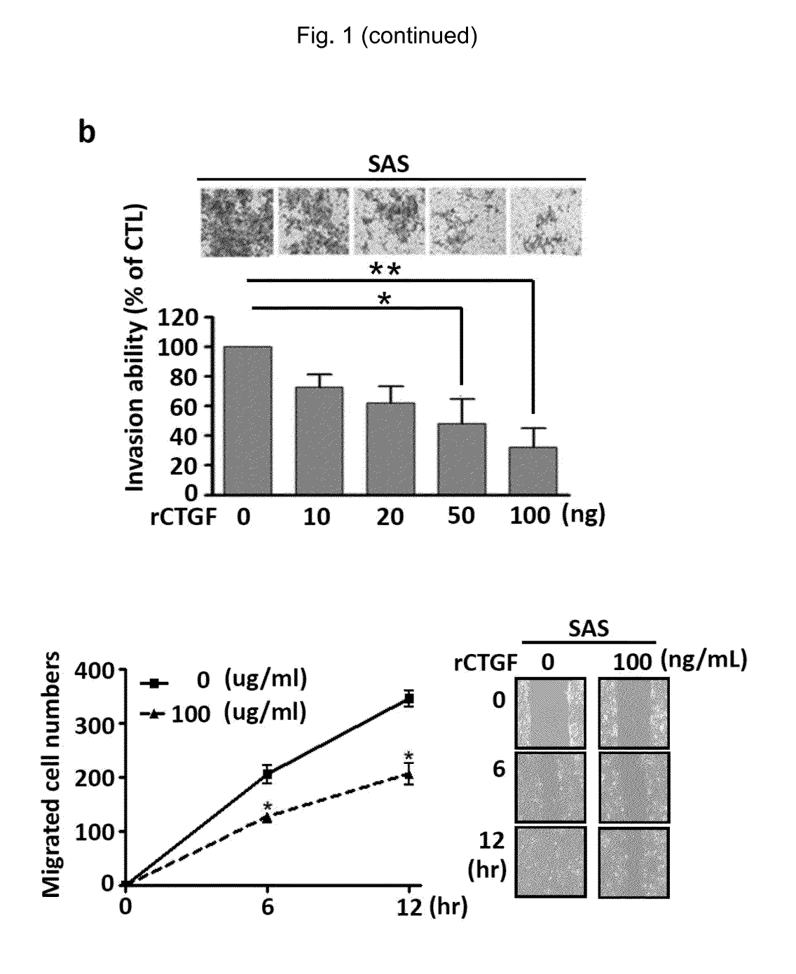

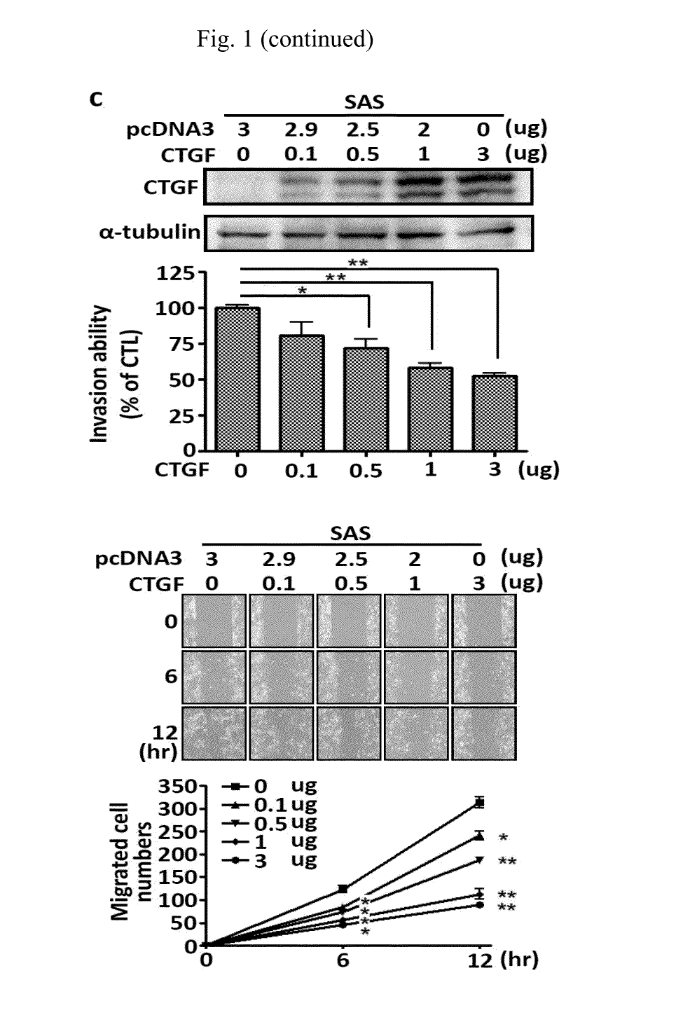

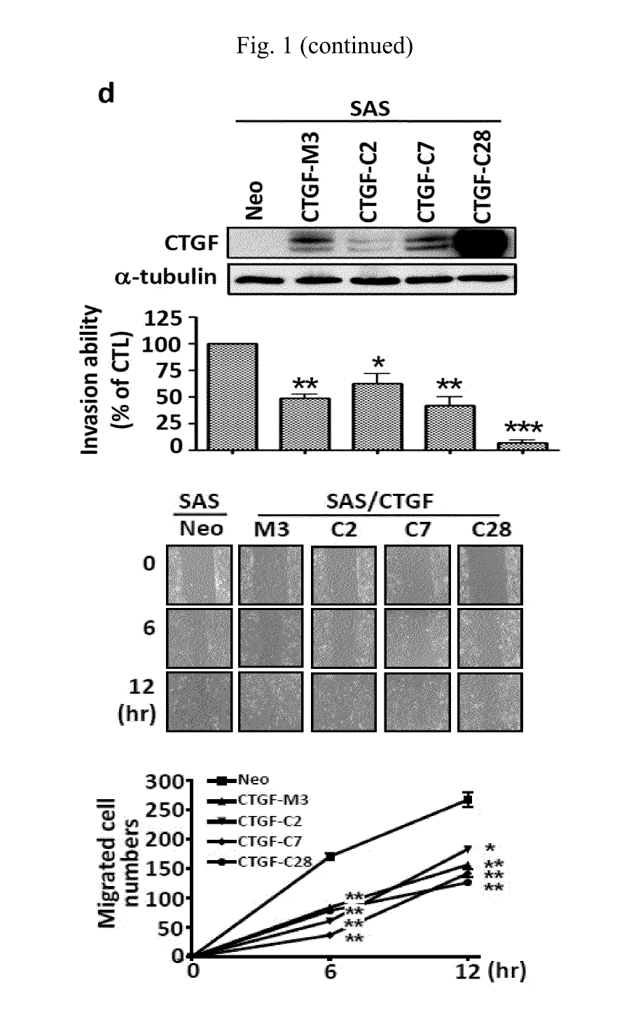

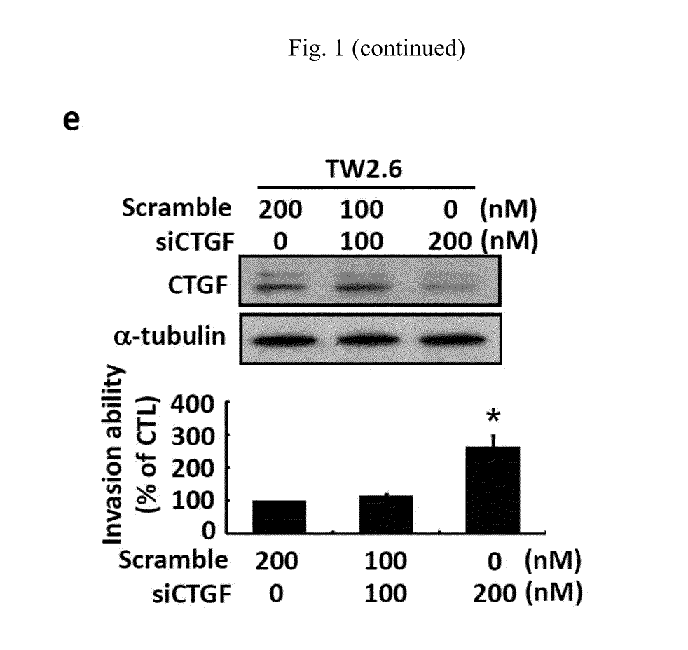

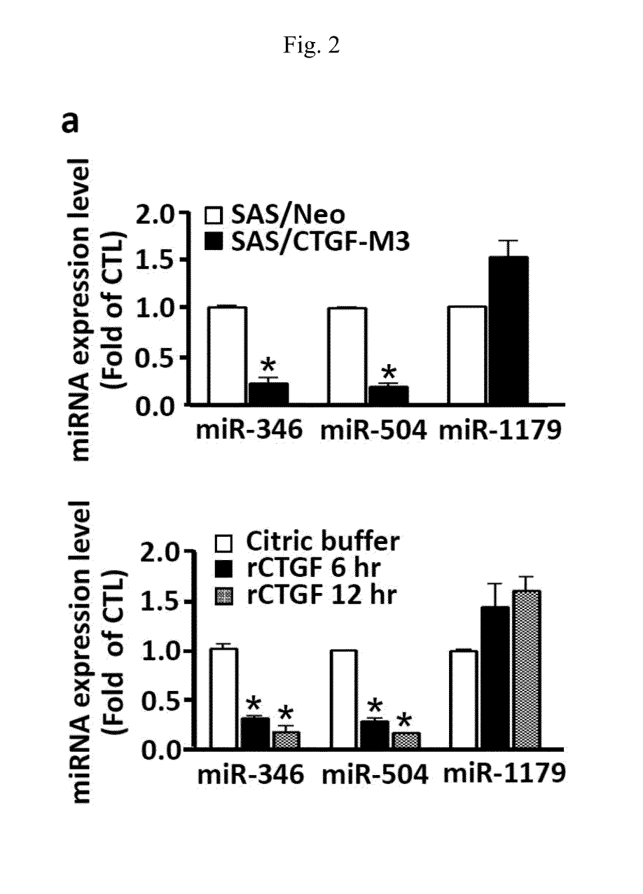

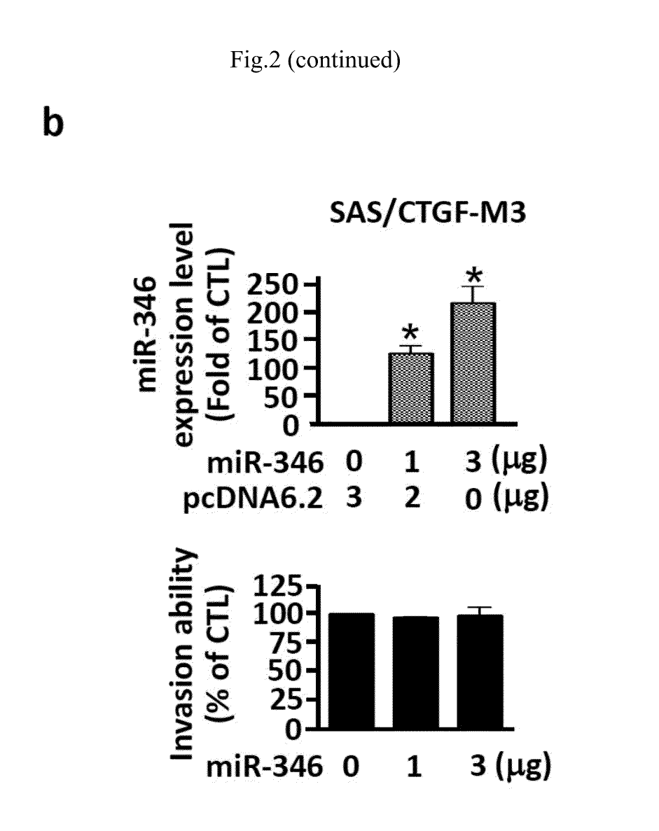

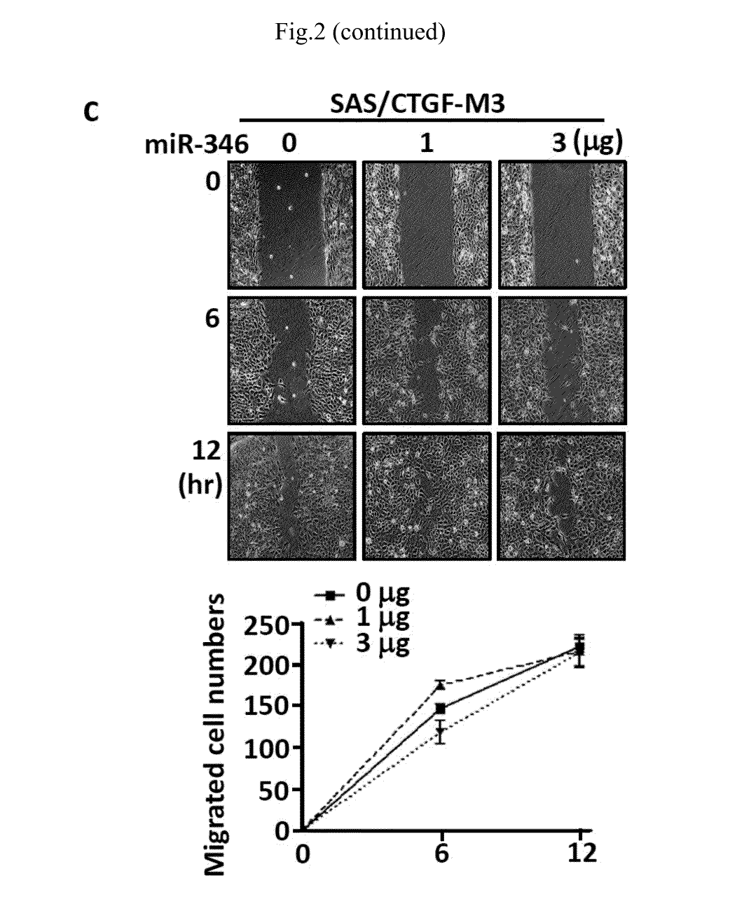

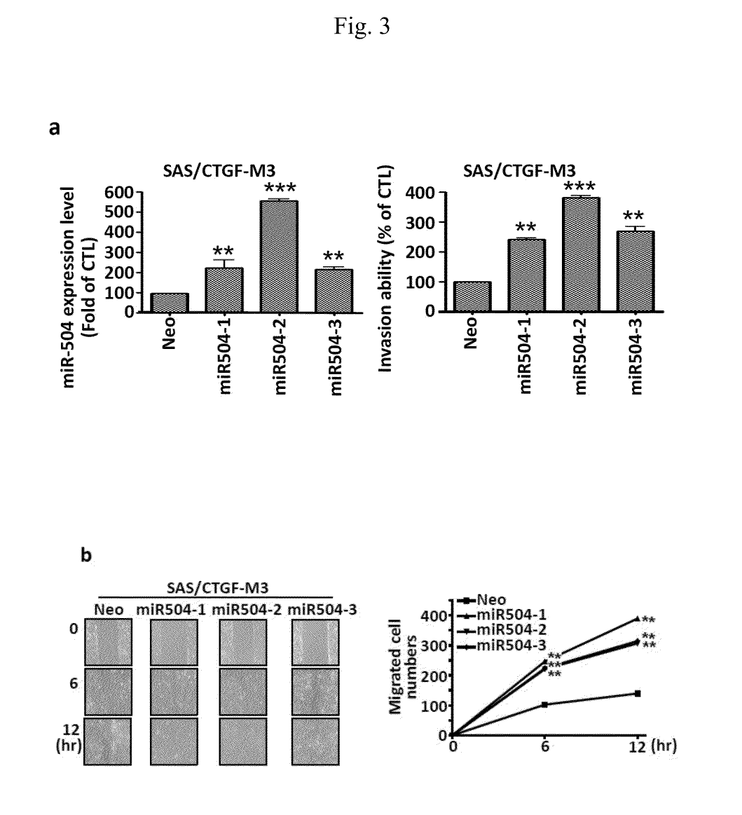

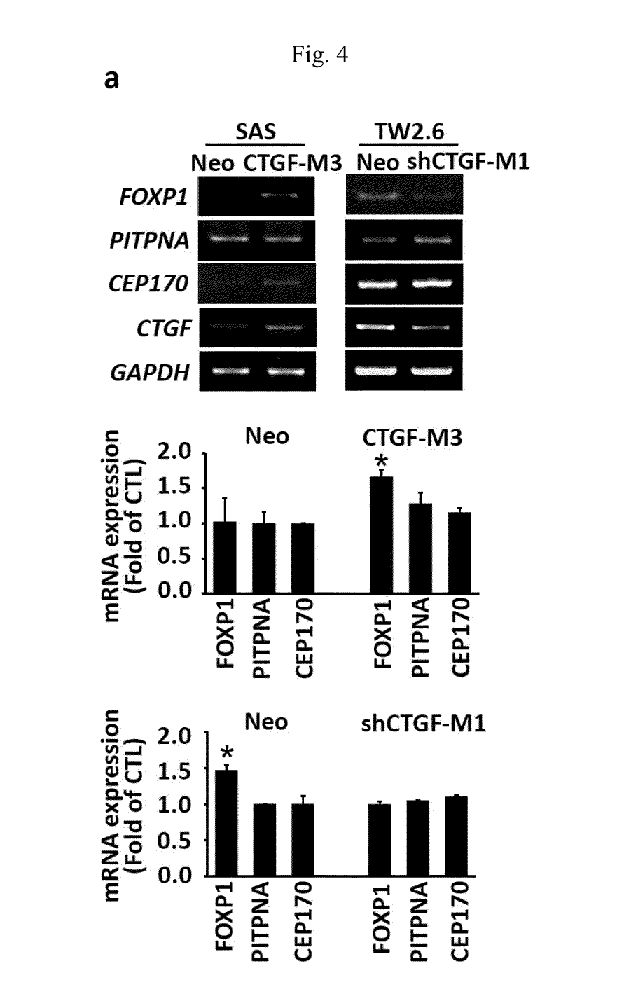

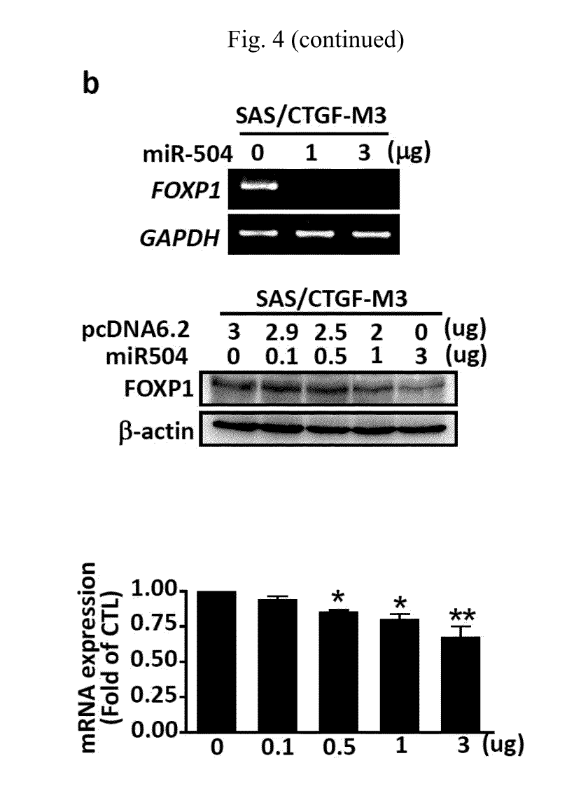

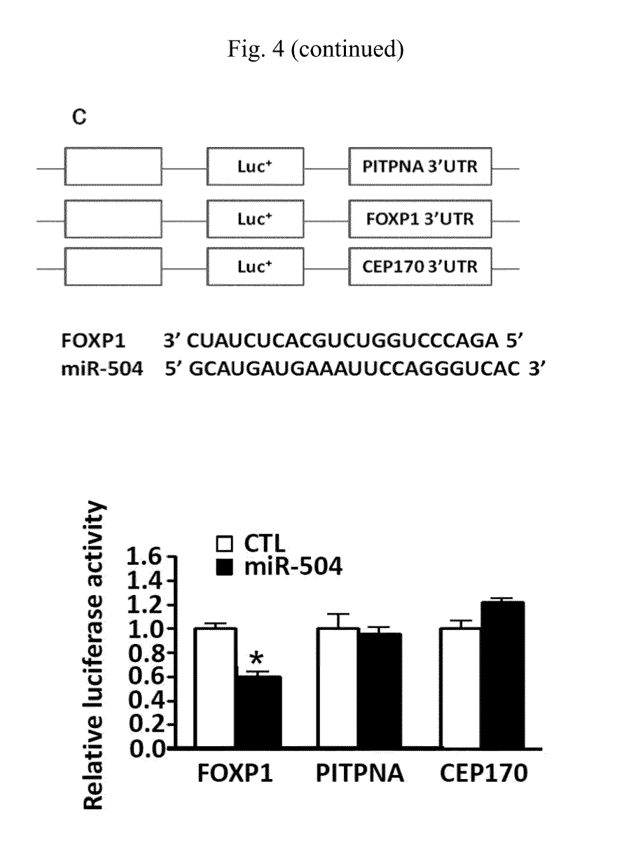

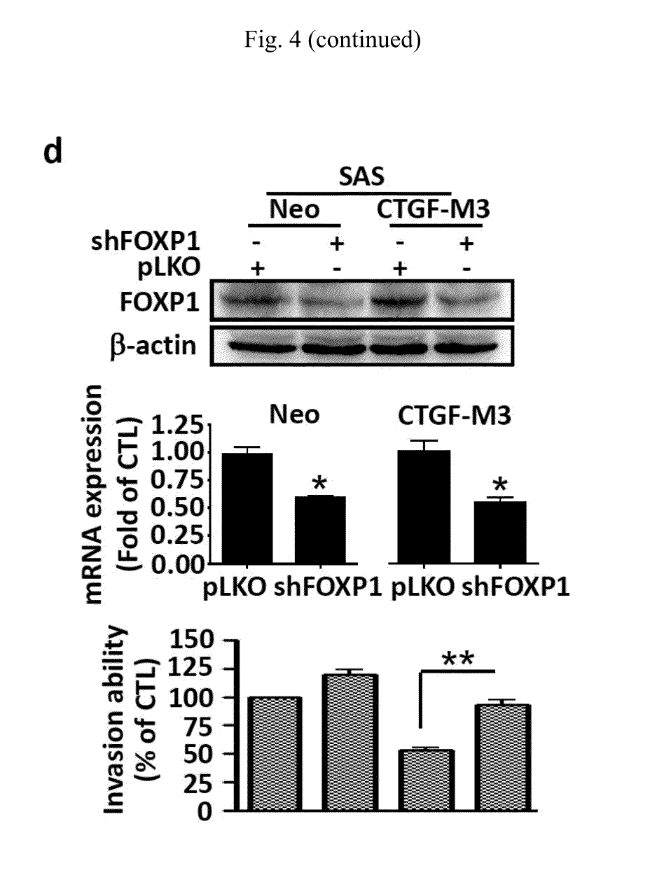

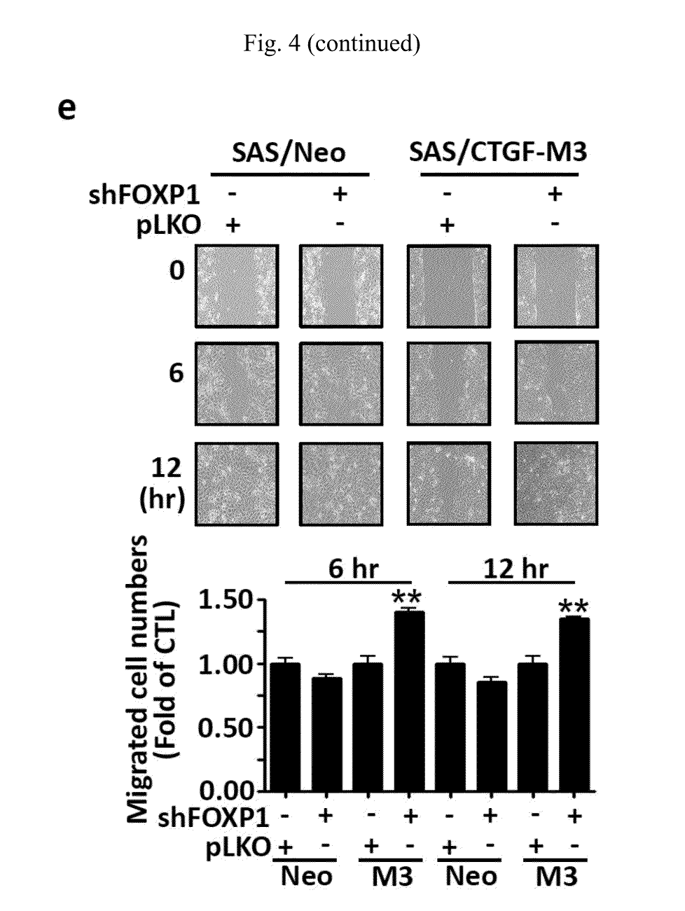

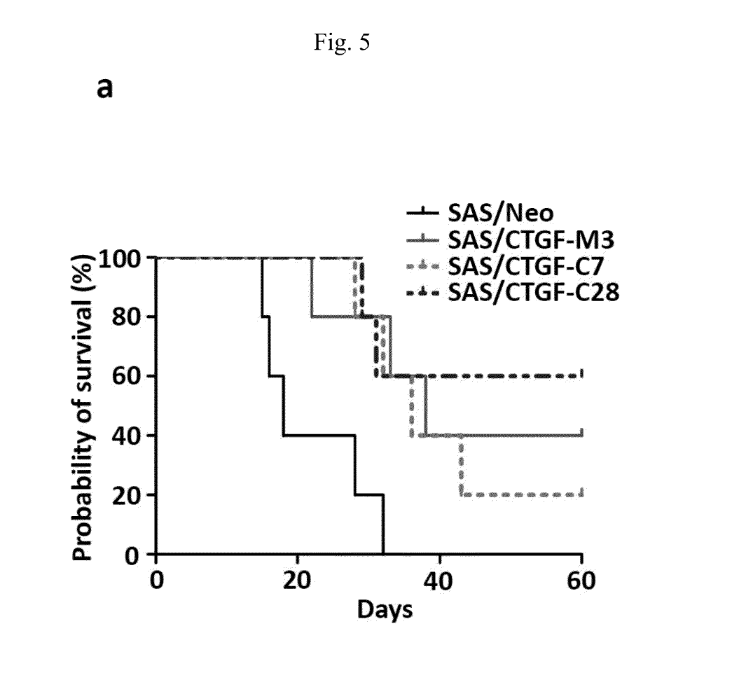

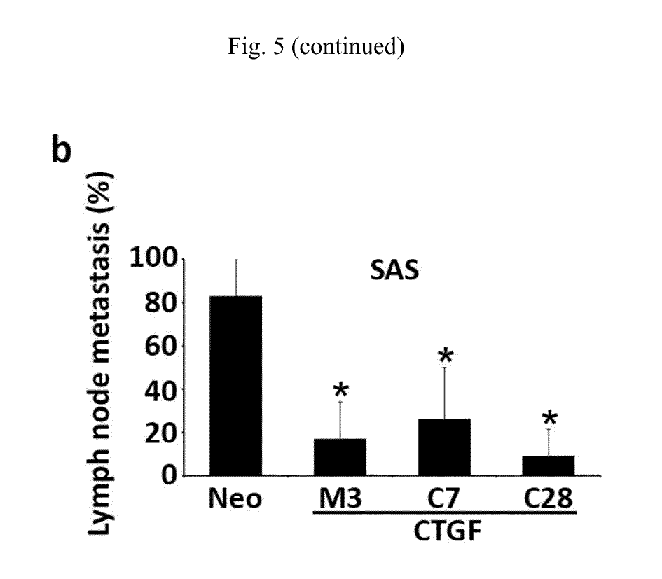

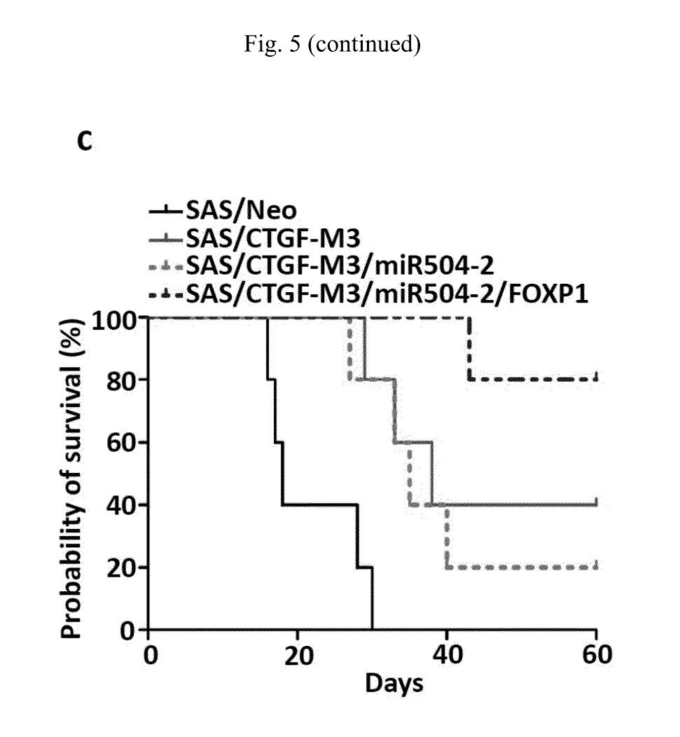

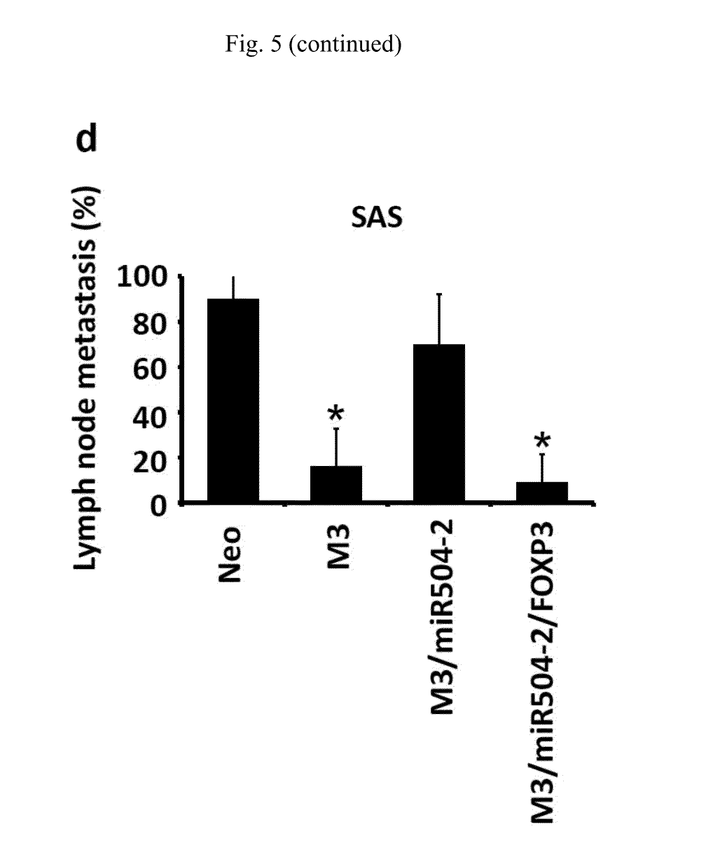

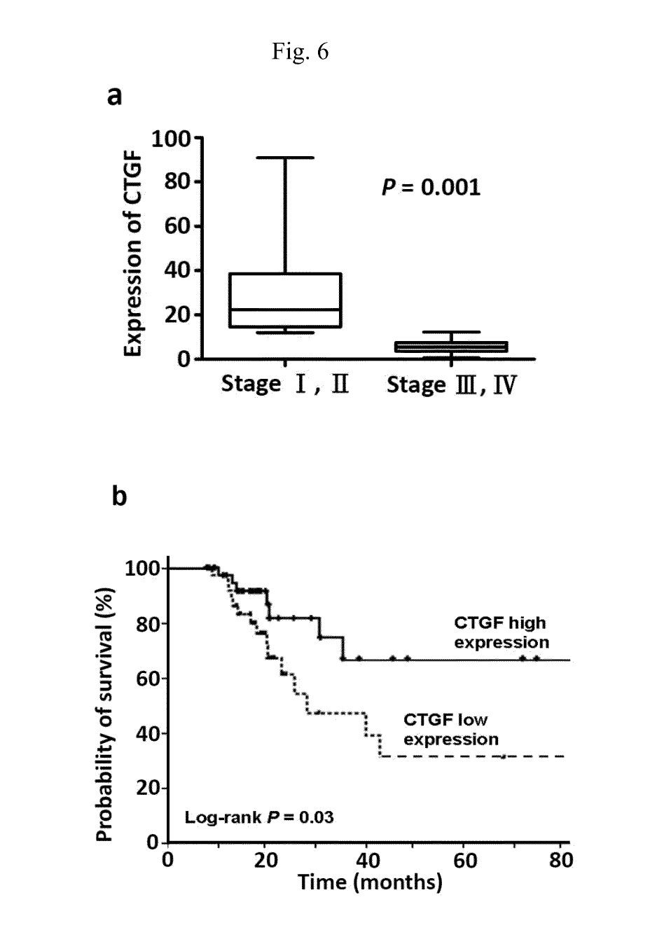

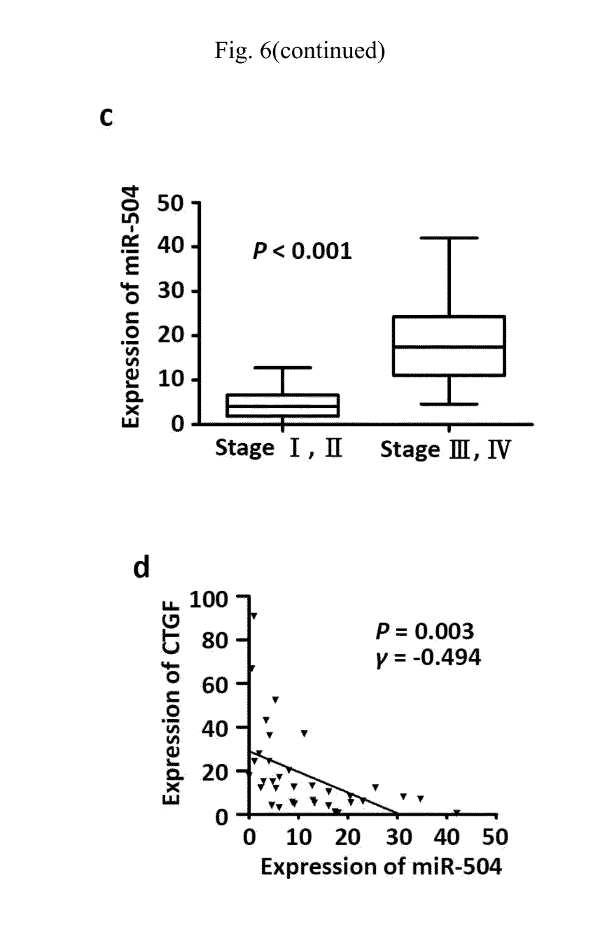

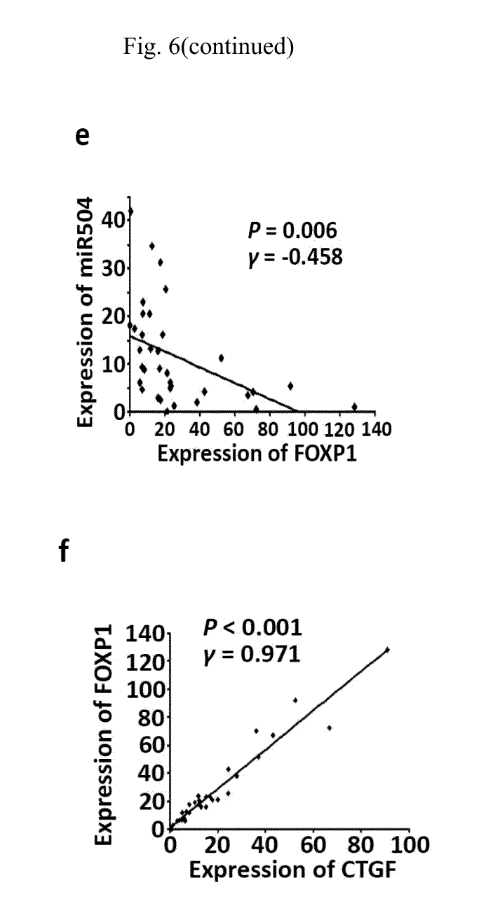

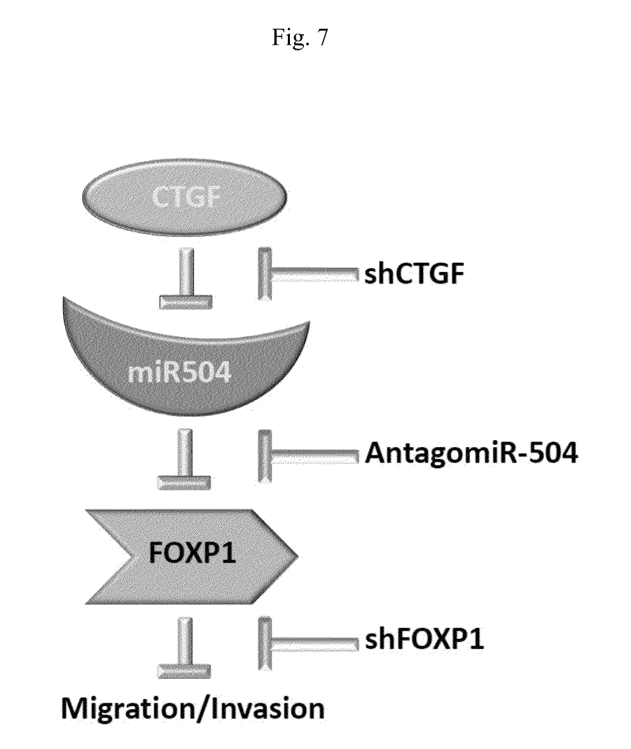

The possible role of CTGF in the invasiveness of OSCC was recognized in the example, where the correlation between invasion ability and CTGF expression in five OSCC cell lines, including TW2.6, CAL-27, HSC3, Ca9-22 and SAS, was examined. It was found that CTGF mRNA and protein were highly expressed in low-invasive cells such as TW2.6, CAL-27 and HSC3 cells, but were almost undetectable in highly invasive Ca9-22 and SAS cells (FIG. 1a).

The data demonstrated that CTGF expression was inversely associated with the metastatic phenotype in human oral cancer cell lines. Thus, we hypothesized that CTGF may have a critical role in oral cancer metastatic progression.

As CTGF is a secreted protein, the recombinant CTGF protein (rCTGF) was used to treat the low-CTGF-producing SAS cells and observed the impact of exogenous CTGF on cellular migration/invasion. rCTGF effectively inhibited SAS cells invasion ability in a dose-dependent manner (FIG. 1b, upper). The migration ability of SAS cells was also significantly decreased by rCTGF treatment (FIG. 1b, lower).

Furthermore, e SAS cells were transiently transfected with various concentrations of CTGF expression plasmid and then determined the invasion and migration abilities, and a dose-dependent decrease in invasion/migration of SAS cells by CTGF was shown (FIG. 1c).

To further elucidate the effect of CTGF in oral cancer cell invasion, CTGF stable transfectants in SAS cells were established. After proper selection by antibiotics, three CTGF-overexpressed clones (SAS/CTGF-C2, C7 and C28), a mixed population (SAS/CTGF-M3) and vector control (SAS/Neo) cells were established (FIG. 1d, upper panel). A reduced invasion/migration was shown in these CTGF stable clones (FIG. 1d, second and lower panel).

In contrast, short-interference RNA-mediated knockdown of CTGF in high-CTGF-producing TW2.6 cells showed notably increased invasive ability of about 1.2˜2.5-fold compared with the scrambled control (FIG. 1e). Taken together, these results indicated that CTGF significantly suppressed invasion and migration abilities in oral cancer cells.

### Example 2

### Identification of Putative Downstream miRNA(s) Mediating CTGF-Induced Migratory Inhibition

To identify the mechanism(s) of CTGF-mediated inhibition in oral cancer progression, we utilized the miRNA microarray to compare the profiles of SAS/CTGF-M3 and SAS/Neo clones. Among the significantly regulated miRNAs, miR-504 and miR-346 were two of the most downregulated, and miR-1179 was upregulated in response to

CTGF overexpression. Using quantitative reverse transcriptase (RT)-PCR analysis, these changes of miRNAs expression in SAS/CTGF-M3 versus control cells were confirmed. To avoid the adaptation effect in stable transfectants, rCTGF was used to treat SAS cells.

A significant suppression of miR-346 and miR-504 by CTGF was shown both in stable transfection and exogenous treatment system; however, only a marginal increase in miR-1179 was found in CTGF-transfected/treated cells (FIG. 2a). These results suggested that miR-346 and miR-504 may be putative CTGF downstream miRNAs participating in CTGF-induced phenotype of OSCC cells.

### Example 3

### Determination for the Function of miRNAs and CTGF in Tumor Migration/Invasion

To identify the key downstream miRNA(s) involved in CTGF-inhibited oral cancer cell migration/invasion, miR-346 or miR-504 was transfected into SAS/CTGF-M3 clone and assayed for invasion ability. As shown in FIG. 2b, after confirming the miR-346 expression level in SAS/CTGF-M3, It was found that there was no significant difference in invasion ability between miR-346-overexpressed SAS/CTGF-M3 and control cells.

Consistently, wound-healing assay also showed no significant difference in miR-346-transfected cells (FIG. 2c). Therefore, although miR-346 was decreased in CTGF-overexpressed clone, it may not be involved in the migration/invasion inhibitory mechanism by CTGF.

To clarify the potential role of miR-504 in CTGF-inhibited migration/invasion, miR-504 expressing plasmids and control vectors were transfected into low-invasive SAS/CTGF-M3 transfectant to check the functional outcome. Transient-transfected miR-504 resulted in a marked enhancement of invasion ability after 48-h transfection (data not shown).

Furthermore, in CTGF transfectant SAS/CTGF-M3, three miR-504-overex-pressed mixture populations (SAS/M3-miR-504-1, SAS/M3-miR-504-2 and SAS/M3-miR-504-3) and a vector control clone (SAS/M3-Neo) were generated for subsequent experiments (FIG. 3a, left panel). Consistently, overexpression of miR-504 in SAS/M3 cells significantly increased invasion (FIG. 3a, right panel) and migration abilities (FIG. 3b). These results indicated that miR-504 is the crucial miRNA of CTGF-regulated invasion/migration pathway in oral cancer.

### Example 4

### Determination for the Downstream Target Gene in CTGF-Regulated miR-504 Signalling Pathway

To identify the mechanism of miR-504-involved OSCC invasiveness, possible downstream genes were searched using bioinformative screening analysis of miRNA target databank: TargetScan, MIRADA and EIMMo, which compute optimal sequence complementarity between a set of mature miRNAs and a given mRNA using a weighted dynamic programming algorithm.

From these three databanks overlapping between the predicted targets of miR-504, FOXP1, PITPNA and CEP170 were ranked as the most probable targets of miR-504. Then. their mRNA levels were determined in previously established CTGF stable clones. The result showed that FOXP1 was the only one with a corresponding change to CTGF manipulation in OSCC cells, that is, overexpression of CTGF in SAS cells upregulated FOXP1, and repression of CTGF in TW2.6 cells attenuated FOXP1 (FIG. 4a).

To further verify the direct effect of miR-504 on FOXP1 regulation, we transiently transfected indicated concentrations of the miR-504 expression plasmid into SAS/CTGF-M3, and FOXP1 expression was inhibited by miR-504 in a dose-dependent manner (FIG. 4b). A 3′ UTR reporter assay in HEK-293T cells also showed that among these three putative targets, only the FOXP1 reporter was significantly repressed by miR-504 (FIG. 4c). A similar result was obtained from the same experiment performed in SAS cells (data not shown), suggesting the FOXP1 as an important target of miR-504 in CTGF regulation machinery.

To evaluate the invasion ability and the mechanistic link between CTGF, miR-504 and FOXP1, FOXP1 expression was knock-downed by transfecting short-hairpin RNA (shFOXP1) into SAS/CTGF-M3 cells and SAS/Neo control cells and observed the migration and invasion ability in these clones.

The results showed that loss of FOXP1 increased invasion (FIG. 4d) and migration (FIG. 4e) of SAS/CTGF-M3 cells, compared with SAS/Neo control cells. Collectively, these data support that suppression of miR-504 by CTGF, resulting in upregulation of FOXP1 expression contributes to CTGF-mediated inhibition of OSCC invasiveness.

### Example 5

### Determination for the Significance of CTGF-miR504-FOXP1 Axis? In Vivo and in OSCC Patients

To confirm the effect of CTGF overexpression in OSCC progression and metastasis in vivo, CTGF stable transfectants (SAS/CTGF-M3, SAS/CTGF-C7 and SAS/CTGF-C28) and a vector control clone (SAS/Neo) were injected into the buccal mucosa of SCID mice. All mice implanted with SAS/Neo were moribund within 40 days.

As shown in FIG. 5a, the overall survival was significantly longer in SAS/CTGF groups than in SAS/Neo group (Neo versus CTGF-M3, P=0.0127; Neo versus CTGF-C7, P=0.0018; Neo versus CTGF-C28, P=0.0198). Moreover, the average number of metastatic lymph nodes in mice injected with CTGF-overexpressed clone was markedly reduced by >50% compared with that of SAS/Neo control group (FIG. 5b).

To further investigated the impact of CTGF-miR-504-FOXP1 axis in OSCC metastasis, we generated the stable clones by reconstitution of miR-504 (SAS/CTGF-M3/miR-504-2) or co-expression of miR-504 and FOXP1 (SAS/CTGF-M3/miR-504-2/FOXP1) in CTGF transfectants, and performed the orthotopic in vivo experiments. The results showed that reconstitution of miR-504 in SAS/CTGF-M3 correlated with a trend of shortened survival of mice (FIG. 5c; P=0.6115, log-rank test), and abrogated the metastasis-suppressing effect by CTGF (FIG. 5d). Co-expression of miR-504 and FOXP1 in SAS/CTGF-M3 restored the suppression of metastasis by CTGF and extended the mice's survival (FIGS. 5c and d).

Finally, in the example, the clinical importance of CTGF-miR-504-FOXP1 axis in OSCC patients was investigated. Quantitative real-time RT-PCR analysis of CTGF, miR-504 and FOXP1 was performed in 85 OSCC patient tumor samples. The result showed that that high CTGF mRNA expression was significantly associated with an early TNM stage (P=0.001, FIG. 6a), and patients with a low CTGF expression were associated with a poorer prognosis (FIG. 6b). Meanwhile, a higher miR-504 expression was correlated with an advanced clinical pathological TNM stage (P<0.001, FIG. 6c). A reverse correlation between CTGF and miR-504 (FIG. 6d), miR-504 and FOXP1 (FIG. 6e), and a positive correlation between CTGF and FOXP1 (FIG. 6f) were also demonstrated.

According to the results, a novel and effective therapy for OSCC metastasis based on such a novel CTGF-mediated signalling pathway model is thus provided: CTGF represses miR-504 expression, which results in the augmentation of FOXP1 expression and leads to the attenuation of migratory ability and invasiveness of OSCC. Enhancement of CTGF expression or antagonist against miR-504 will reconstitute the FOXP1 expression and inhibit OSCC migration/invasion (FIG. 7). These data serve as a foundation for future development designed to explore further action of CTGF and miRNA expression in OSCC cancer progression. This secreted protein appears to can be a potential candidate molecular protein drug to treat human oral carcinoma by modulating its invasion ability.

In summary, above-mentioned embodiments provide a new composition for modulating invasion ability of a tumor. The composition functions according to a novel model that the secreted cytokine or activator such as CTGF can attenuate cellular invasion/migration of tumor (e.g. OSCC) via a miRNA-mediated pathway. Here, miRNAs such as miR-504 play the critical role in promoting metastasis through regulation of its downstream target such as a novel target, FOXP1 gene. A new treating method using the composition is provided as well. Meanwhile, a detecting kit and a detecting method based on the novel model are also provided.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A composition for modulating invasion ability of a tumor, comprising:
an effective amount of an activator for a miRNA-mediated pathway or an effective amount of a modulating member in the miRNA-mediated pathway, and
wherein the miRNA-mediated pathway is regulated by at least one miRNA selected from the group consisting of miR-346, miR-504 and miR-1179.

2. The composition of claim 1, wherein the activator is a CTGF gene product.

3. The composition of claim 2, wherein the CTGF gene product is a transcript, a polypeptide, or a protein.

4. The composition of claim 1, wherein the modulating member is a miRNA which mediates the miRNA-mediated pathway and is selected from the group consisting of miR-346, miR-504 and miR-1179.

5. The composition of claim 1, wherein the modulating member is a miRNA downstream target gene product involved in the miRNA-mediated pathway selected from the group consisting of FOXP1, PITPNA and CEP170.

6. The composition of claim 1, wherein the tumor is oral cancer.

7. The composition of claim 6, wherein the oral cancer is oral squamous cell carcinoma (OSCC).

8. The composition of claim 7, wherein the miRNA-mediated pathway is a miR-504 pathway, and the modulating member is miR-504, a gene product of FOXP1 or a combination thereof.

9. A method for treating or preventing tumor invasion in a subject in need thereof, the method comprising,
administering an effective amount of the composition according to claim 1.

10. A method for detecting the invasive ability of a tumor in a subject, comprising the steps of:
(a). determining an expression level of an activator for a miRNA-mediated pathway or a modulating member in the miRNA-mediated pathway in a sample obtained from the subject, wherein the miRNA-mediated pathway is regulated by at least one miRNA selected from the group consisting of miR-346, miR-504 and miR-1179;
(b). comparing the expression level of the activator or the modulating member in the sample with a standard level of the activator or the modulating member in the sample; and
whereby a difference between the expression level and the standard level indicates the invasive ability of the tumor.

11. The method of claim 10, wherein the activator is a CTGF gene product.

12. The method of claim 11, wherein the CTGF gene product is a transcript, a polypeptide, or a protein.

13. The method of claim 10, wherein the modulating member is a miRNA which mediates miRNA-mediated pathway and is selected from the group consisting of miR-346, miR-504 and miR-1179.

14. The method of claim 10, wherein the modulating member is a miRNA downstream target gene product involved in the miRNA-mediated pathway selected from the group consisting of FOXP1, PITPNA and CEP170.

15. The method of claim 10, wherein the tumor is oral cancer.

16. The method of claim 15, wherein the oral cancer is oral squamous cell carcinoma (OSCC).

17. The method of claim 16, wherein the miRNA-mediated pathway is a miR-504/FOXP1 pathway, and the modulating member is miR-504 or a gene product of FOXP1 or a combination thereof.

18. A kit for detecting the invasive ability of a tumor in a subject, comprising:
an activator for a miRNA-mediated pathway or a modulating member in the miRNA-mediated pathway; and
an instruction for detecting the invasive ability of a tumor in a subject according to claim 10,
wherein the miRNA-mediated pathway is regulated by at least one miRNA selected from the group consisting of miR-346, miR-504 and miR-1179.

19. The kit of claim 18, wherein the activator is a CTGF gene product.

20. The kit of claim 19, wherein the CTGF gene product is a transcript, a polypeptide, or a protein.

21. The kit of claim 18, wherein the modulating member is a miRNA which mediates the miRNA-mediated pathway and is selected from the group consisting of miR-346, miR-504 and miR-1179.

22. The kit of claim 18, wherein the modulating member is a miRNA downstream target gene product involved in the miRNA-mediated pathway selected from the group consisting of FOXP1, PITPNA and CEP170.

23. The kit of claim 18, wherein the tumor is oral cancer.

24. The kit of claim 23, wherein the oral cancer is oral squamous cell carcinoma (OSCC).

25. The kit of claim 24, wherein the miRNA-mediated pathway is a miR-504/FOXP1 pathway, and the modulating member is miR-504 or a gene product of FOXP1 or a combination thereof.


SEQUENCE LISTING (10 sequences)

SEQ ID NO: 1 - DNA, 23 bp, Artificial Sequence
  RT-PCR primer sequence
gcttaccgactggaagacacgtt

SEQ ID NO: 2 - DNA, 22 bp, Artificial Sequence
  RT-PCR primer sequence
tcatgccatgtctccgtacatc

SEQ ID NO: 3 - DNA, 20 bp, Artificial Sequence
  RT-PCR primer sequence
cctgtggggagcggggatga

SEQ ID NO: 4 - DNA, 20 bp, Artificial Sequence
  RT-PCR primer sequence
ctgagtggcagcaccagccc

SEQ ID NO: 5 - DNA, 21 bp, Artificial Sequence
  RT-PCR primer sequence
acaggtgcagggcatgcttca

SEQ ID NO: 6 - DNA, 26 bp, Artificial Sequence
  RT-PCR primer sequence
tctactccaaacacaacagcttggtc

SEQ ID NO: 7 - DNA, 24 bp, Artificial Sequence
  RT-PCR primer sequence
gccgattcattccacgcagcagta

SEQ ID NO: 8 - DNA, 23 bp, Artificial Sequence
  RT-PCR primer sequence
ccacacccgttatcgcagagcac

SEQ ID NO: 9 - DNA, 19 bp, Artificial Sequence
  RT-PCR primer sequence
gaaggtgaaggtcggagtc

SEQ ID NO: 10 - DNA, 19 bp, Artificial Sequence
  RT-PCR primer sequence
caggaggcattgctgatga